(19) United States
(12) Patent Application Publication
(10) Pub. No.: US 2024/0252768 A1
(43) Pub. Date: Aug. 1, 2024

(54) MEDICAMENT DISPENSERS, SYSTEMS AND METHODS

(71) Applicant: Bespak Europe Limited, King's Lynn (GB)

(72) Inventors: Woo Suk Kim, Cambridge (GB); Rachel Koppelman, Cambridge (GB)

(21) Appl. No.: 18/565,071

(22) PCT Filed: May 17, 2022
(86) PCT No.: PCT/EP2022/063323

§ 371 Date: Nov. 28, 2023

(30) Foreign Application Priority Data
Jun. 10, 2021 (GB) 2108288.8

(51) Int. Cl. A61M 5/36; A61M 5/315

(52) U.S. Cl. CPC A61M 5/36; A61M 5/31525

(57) ABSTRACT

A medicament dispenser (1) comprising: a body (10) comprising an inlet (11) configured to interface with a syringe (70) and an outlet (15) configured to interface with a needle (60); a dosing chamber (20) within said body comprising a dosing chamber inlet (21) fluidly connected to the inlet (11) of the body (10) and a dosing chamber outlet (22) fluidly connected to the outlet (15) of the body (10), the dosing chamber (20) being configured to contain a fixed volume of medicament (75) when full; and a piston (40) configured to be movable within the dosing chamber (20) to deliver the fixed volume of medicament (75) out of the outlet (15) of the body (10).

The present disclosure relates to medicament dispensers, medicament dispensing systems and associated methods.

Background to the Disclosure With injection devices such as syringes there is a need to ensure that the medicament contained in the syringe can be delivered to a patient in a manner that avoids infection. Consequently, significant effort and expense can be entailed in ensuring that the syringe and its contents are maintained in a sterile state until the point of use.

In addition, with syringes there is a need to ensure that the medicament can be delivered with a high level of dose accuracy. This can be particularly important where the volume of the dose being delivered is small. For example, with ocular injections, in particular intravitreal injections, the dosage volume may be as little as 50 μl or less. While it is known to provide syringes with graduated markings on the barrel of the syringe, the use of such markings relies on the visual acuity and manual dexterity of the medical practitioner. Such markings can also be difficult to use accurately when the dosage volume is small.

Another potential problem when using syringes to inject medicament is the potential presence of air bubbles in the syringe prior to use. Air bubbles may affect the accuracy of the dosage injected and can also cause complications for the patient. For example, in the case of ocular injections, injecting air bubbles into the eye may lead to complications including temporary vision impairment and increased intraocular pressure. Consequently, it is known to try and remove air bubbles from a syringe prior to use by performing a ‘purging’ step wherein the syringe is orientated with the needle vertically upwards, the syringe is flicked or knocked to move air bubbles towards an upper end of the medicament-containing chamber within the syringe barrel, and the piston of syringe is then advanced within the barrel by operation of the plunger until the air and then liquid medicament is discharged from the needle tip. However, such purging of syringes can be ineffective in removing all air bubbles, in particular where the liquid medicament is viscous, the air bubbles are small compared to the internal diameter of the syringe barrel and/or the liquid medicament has a high wetness. With high viscosity liquids air bubbles may only rise slowly and therefore not be effectively purged. When the air bubbles are small compared to the syringe barrel diameter the liquid medicament has a tendency to flow around and by-pass the air bubbles rather than pushing the air bubbles towards the syringe outlet. When the liquid medicament has a high wetness air bubbles will have a tendency to remain held in place against the wall of the syringe barrel due to surface tension effects.

In addition, purging of syringes can be difficult to carry out effectively where the available pre-stroke of the piston is small. By “pre-stoke” is meant the available stroke length of the piston that is available until the piston reaches the marked graduation for the intended dosage volume. Where the pre-stroke is small there may be insufficient movement available to completely discharge all air bubbles out of the needle tip. In such cases there is a risk that air bubbles will remain and potentially be injected into the patient and/or that the practitioner will continue the purge by pushing the piston beyond the graduated marking. This can lead to insufficient dosage volume then being injected, affecting the dose accuracy. While, a partial solution to this problem can be to increase the pre-stroke length of the syringe this is at the cost of increasing the volume of medicament needed in the syringe which can significantly increase the cost of production, especially where the medicament is itself expensive. In addition, it may be undesirable to have a high fill volume of medicament due to the risk of injecting too greater volume into the patient, for example if the purging step is not performed.

It is an object of the present disclosure to provide medicament dispensers, medicament dispensing systems and associated methods that at least partially alleviate some of the problems mentioned above.

### SUMMARY OF THE DISCLOSURE

In a first aspect the present disclosure provides a medicament dispenser comprising:
a body comprising an inlet configured to interface with a syringe and an outlet configured to interface with a needle;
a dosing chamber within said body comprising a dosing chamber inlet fluidly connected to the inlet of the body and a dosing chamber outlet fluidly connected to the outlet of the body, the dosing chamber being configured to contain a fixed volume of medicament when full; and
a piston configured to be movable within the dosing chamber to deliver the fixed volume of medicament out of the outlet of the body.

Advantageously, the medicament dispenser of the present disclosure may alleviate problems with purging of air bubbles from a syringe by providing a separate dosing chamber in a body other than the syringe into which a liquid medicament may be transferred from the syringe. This permits the option of fully stroking the piston of the syringe, i.e. moving/pushing the piston all the way along the syringe barrel until an end face of the piston is at or adjacent the distal end of the syringe barrel. In this way, most if not all air bubbles within the medicament-containing chamber of the syringe will be physically pushed out of the syringe, including sweeping off any air bubbles clinging to the wall of the barrel, by the transiting piston. Thereby the problems of surface tension effects between the air bubbles and the syringe wall and liquid medicament by-passing the air bubbles within the syringe may be overcome. In addition, since purging may be carried out by fully stroking the piston and plunger of the syringe there is no requirement for the medical practitioner to judge when to stop the piston at a correct graduated marking on the syringe barrel. Rather, the dose accuracy may be controlled and assured by the pre-determined and fixed volume of the dosing chamber in the body.

Optionally, the dosage chamber of the body has a volume that is smaller than a volume of the medicament-containing chamber of the syringe, such that on completion of the full stroke of the syringe piston and after a portion of the liquid medicament and any air bubbles are purged out of the outlet of the medicament dispenser, the dosing chamber is left full of substantially air bubble-free liquid medicament. Thereafter, the fixed volume of the liquid medicament in the dosing chamber may be injected by operating the plunger of the medicament dispenser. The plunger may be fully stroked, e.g. fully depressed until the piston of the medicament dispenser is in face-to-face contact with an end face of the dosing chamber at or adjacent the dosing chamber outlet. Consequently, during both the purging of the syringe and the injection of the liquid medicament to the patient, the medical practitioner may only be required to fully stroke each of the plungers of the syringe and the medicament dispenser. All partial stokes of a plunger may be avoided. Consequently, the medical practitioner does not need to make any visual judgements on the degree to move either plunger during purging or injection. Thus, a very reliable dose accuracy may be obtained that is not reliant on the visual acuity or manual dexterity of the medical practitioner.

A range of medicament dispensers as described may be provided which differ one from the other by the volume of their dosing chambers. Thus, a medical practitioner may simply select the appropriate medicament dispenser that has a dosing chamber that matches the desired dose volume for a particular patient.

The medicament dispenser may be rendered sterile by known techniques. In particular, the medicament dispenser may be made from a biocompatible material, such as an engineering plastic, that is compatible with known sterilising processes.

The dosing chamber inlet may be fluidly connected to the inlet of the body by an inlet passage and the dosing chamber outlet may be fluidly connected to the outlet of the body by an outlet passage. The inlet passage and/or the outlet passage may be straight. The use of straight passages may advantageously improve the manufacturability of the medicament dispenser, e.g. by allowing the passages to be formed by injection moulding of the body. In addition, the use of straight passages may improve the ease of sterilising the medicament dispenser by providing a less tortuous fluid transport path through the body for movement of a sterilising fluid or gas.

A dosing chamber axis of the dosing chamber may be angled with respect to an inlet axis of the inlet of the body and/or an outlet axis of the outlet of the body. The angle between the dosing chamber axis and the outlet axis of the body may be between 45 and 85 degrees. The angle between the dosing chamber axis and the inlet axis of the body may be between 45 and 85 degrees.

The inlet passage and the outlet passage may be laterally offset from one another.

When the medicament dispenser is orientated with the outlet of the body pointing vertically upwards, a level of the dosing chamber outlet may be above a level of the dosing chamber inlet. Advantageously, this relative orientation of the dosing chamber outlet and the dosing chamber inlet may improve purging of air bubbles in use by encouraging, due to the buoyancy of the air bubbles in the liquid medicament, the movement of any air bubbles ejected from the syringe into the dosing chamber to transit the dosing chamber and exit the dosing chamber outlet during the purging step.

When the medicament dispenser is orientated with the outlet of the body pointing vertically upwards, the dosing chamber may slope upwardly from a location of the dosing chamber inlet to a location of the dosing chamber outlet. For example, it may slope at a slope angle of 5° to 45° to the horizontal; optionally 20° to 30° to the horizontal. The sloping of the dosing chamber may further improve the movement of air bubbles towards and out of the dosing chamber inlet during the purging step.

The inlet and the outlet of the body may be laterally offset from one another. They may be laterally offset and parallel to one another. Such an arrangement may beneficially accommodate a sloping dosing chamber in a space-efficient body. In addition, it may improve the ergonomics of the medicament dispenser by reducing the overall length, front to back, of the device.

The dosing chamber outlet may be located in a terminal end face of the dosing chamber. The dosing chamber outlet may be located at or adjacent a peripheral edge of the terminal end face. The terminal end face may smoothly transition to the outlet passage. The dosing chamber outlet may comprise a funnel portion inbetween the terminal end face and the outlet passage. Advantageously, such an arrangement may improve the discharge of air bubbles out of the dosing chamber. In addition, such an arrangement may reduce or even substantially eliminate the ullage of the dosing chamber helping to increase the dose accuracy delivered.

The piston may be configured to move within the dosing chamber from a pre-activation position to an activated position to deliver the fixed volume of medicament from the dosing chamber through the outlet. In the pre-activation position an end face of the piston may be located immediately adjacent the dosage chamber inlet. Beneficially, this may reduce any dead space in the dosing chamber, for example avoiding any re-entrant void space that might be prone to capture air bubbles during the purging step.

In the activated position an end face of the piston may be located in face-to-face contact with the terminal end face of the dosing chamber, as discussed above. This may beneficially minimise any dead volume in the dosing chamber and improve dose accuracy.

The dosing chamber may have an internal diameter of less than or equal to 4.0 mm; optionally less or equal to 3.5 mm; optionally less or equal to 3.0 mm. The internal diameter of the dosing chamber may be less than that of the chamber of the syringe that initially contains the medicament. As such, the piston in the dosing chamber of the body may have a smaller diameter, a shorter circumference, and a smaller contact area with the wall of the dosing chamber than the piston of the syringe. Beneficially, this may result in the piston of the dosing chamber having a lower break-off force (the force required to overcome static friction to begin movement of the piston) and a lower sliding friction than that of the piston of the syringe. This may result in improved ease of use for the medical practitioner when operating the plunger.

The dosing chamber may be cylindrical. It may have a circular cross-section in a plane perpendicular to its longitudinal axis. Other cross-section shapes may be provided, such as square or hexagonal. The dosing chamber may be straight. In one arrangement the dosing chamber has the shape (excluding the portions immediately adjacent the dosing chamber inlet and outlet) of a right-circular cylinder.

A volume of the dosing chamber may be between 0.03 ml to 0.06 ml.

The fixed volume of medicament may comprise a single dose of medicament.

In some embodiments the piston may be operatively connected to a plunger that extends outside the body of the medicament dispenser.

The plunger may be user-operable. The plunger may be manually operable. The body and plunger may be sized and configured to enable one-handed holding of the body of the medicament dispenser using two or more digits of one hand and activation of the plunger using one or more digits of the same hand.

The plunger may be angled backwardly towards a location of the inlet of the body. This may improve the ergonomics of the medicament dispenser.

The medicament dispenser may further comprise a plunger lock for preventing accidental activation of the plunger. Advantageously, the plunger lock may ensure that the starting point of the piston of the medicament dispenser is reliably known at the point of injection which may improve the dose accuracy.

The plunger lock may comprise a detent on or operably-connected to the plunger that catches on the body. The detent may be movable from a catching orientation to a non-catching orientation. Alternatively, the detent may be removable from the plunger. In one example, the detent may comprise a tear-off element.

In some embodiments the medicament dispenser may comprise a source of motive power for moving the piston on demand. The source of motive power may comprise a source of stored energy. A trigger may be provided for controlling the application of stored energy from the source of motive power to the piston. The source of motive power may, for example, comprise a spring element and/or a propellant.

The inlet of the body may comprise a Luer lock for interfacing with a syringe. The internal dead volume of the inlet of the body when the syringe is interfaced may be minimised, for example by configuring the shape and size of the inlet of the body to closely match the shape and size of the distal end of the syringe. Other, known syringe interfaces may also be supported by appropriate configuration of the inlet of the body.

The medicament dispenser may further comprise a needle fluidly connected to the outlet of the body. The needle may be provided integral with the outlet of the body. Alternatively the outlet of the body may be configured to receive and retain an initially-separate needle. The needle may then be non-detachable.

In a second aspect the present disclosure provides a medicament dispensing system comprising the medicament dispenser of the first aspect and a syringe containing medicament, the syringe being connectable to the inlet of the body.

The medicament dispensing system may further comprise a needle, the needle being connectable to the outlet of the body.

The syringe may comprise a medicament-containing chamber having an internal diameter that is larger than an internal diameter of the dosing chamber of the medicament dispenser.

In a third aspect the present disclosure provides a method for purging air bubbles from a liquid medicament initially contained in a syringe of the type having a medicament-containing chamber having an outlet located at one end and a syringe piston for discharging the liquid medicament, the method comprising the steps of:
a) fluidly attaching the outlet of the syringe to an inlet of a medicament dispenser; and
b) operating the syringe piston through its full stroke to displace liquid medicament from the medicament-containing chamber out of the outlet of the syringe and into, through and out of a fluid transport path of the medicament dispenser, said fluid transport path comprising the inlet of the medicament dispenser, an internal dosage chamber and an outlet of the medicament dispenser;
wherein the internal dosage chamber has a volume that is smaller than a volume of the medicament-containing chamber, such that on completion of the full stroke of the syringe piston a portion of the liquid medicament and a quantity of air bubbles are purged out of the outlet of the medicament dispenser, and the internal dosing chamber is left full of substantially air bubble-free liquid medicament.

The internal dosage chamber may have an internal diameter that is smaller than an internal diameter of the medicament-containing chamber.

During step b) the medicament dispenser may be orientated so that the outlet points vertically upwards and the internal dosing chamber slopes upwardly from a location of a dosing chamber inlet to a location of a dosing chamber outlet.

The fluid transport path may comprise a tortuous path having at least two turns such that the inlet and outlet of the medicament dispenser are laterally-offset from one another.

In a fourth aspect the present disclosure provides a method of manufacturing the medicament dispenser of the first aspect or the medicament dispensing system of second aspect, wherein the body is formed by injection moulding.

As would be appreciated by a person skilled in the art, the syringe may be a wide variety of types of syringe suitable for delivering a medicament to a patient. The syringe may be configured for use with a separate medicament vial or may be a pre-filled syringe (PFS). The syringe may contain a single dose of medicament or multiple doses. The syringe may comprise a glass or plastic barrel. The syringe may be configured for use with a needle that may be integral with the syringe barrel or couplable with the syringe barrel. The syringe may be interfaced with the medicament dispenser of the present disclosure with or without a needle being fitted to the syringe barrel.

The syringe may be configured, for example, for enteral or parenteral injection. For example, for intravenous, intramuscular, subcutaneous, intradermal, intratympanic, intra-articular, intraocular or intravitreal injection.

### BRIEF DESCRIPTION OF THE DRAWINGS

Aspects and embodiments of the present disclosure will now be described, by way of example only, with reference to the accompanying drawings, in which:

FIG. 1 is a side elevation of a medicament dispenser according to the present disclosure fitted with a needle;

FIG. 2 is a perspective view from the front of the medicament dispenser of FIG. 1;

FIG. 3 is a perspective view from the rear of the medicament dispenser of FIG. 1;

FIG. 4 is a cross-sectional view of a medicament dispensing system comprising the medicament dispenser of FIG. 1, a needle and a syringe;

FIG. 5 is a cross-sectional view of the medicament dispensing system of FIG. 4 orientated to carry out a purging step;

FIG. 6 is a cross-sectional view of the medicament dispensing system of FIG. 4 after carrying out the purging step;

FIG. 7 is a cross-sectional view of the medicament dispensing system in a pre-activation position;

FIG. 8 is a cross-sectional view of the medicament dispensing system in an activated position; and

FIG. 9 is an enlarged cross-sectional view of a terminal face of a dosing chamber of the medicament dispenser.

### DETAILED DESCRIPTION

The skilled reader will recognise that one or more features of one aspect or embodiment of the present disclosure may be combined with one or more features of any other aspect or embodiment of the present disclosure unless the immediate context teaches otherwise.

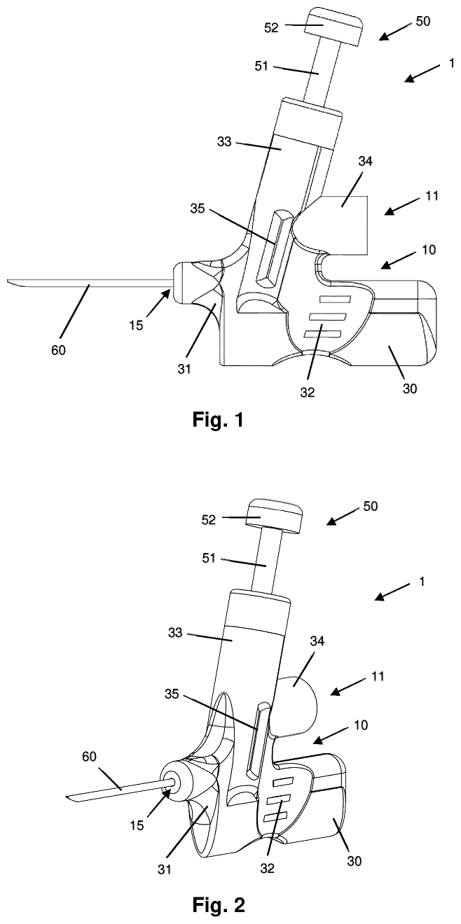

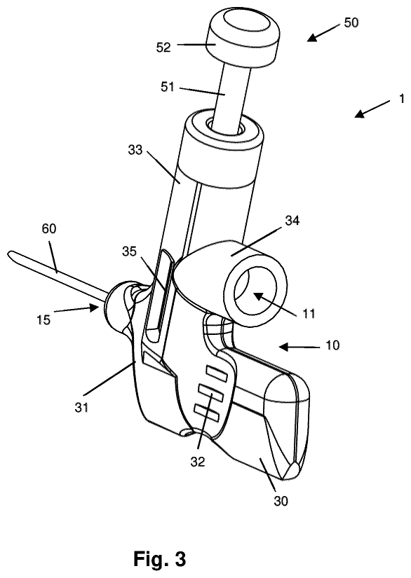

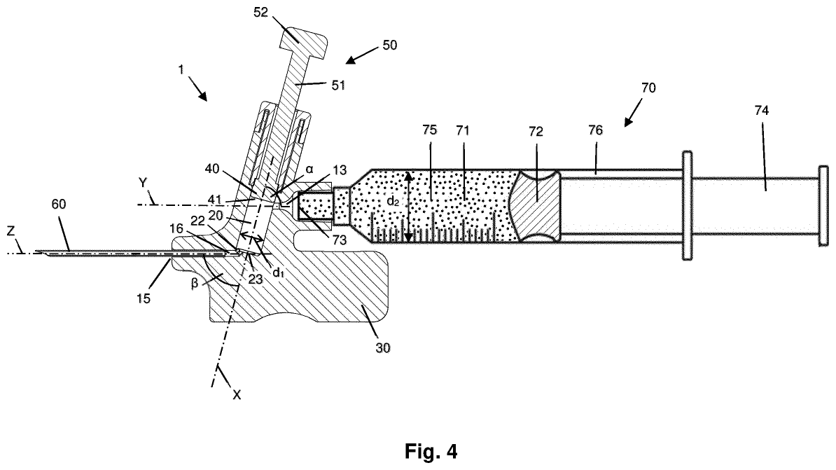

An example apparatus and method in accordance with the present disclosure will now be described with reference first to FIGS. 1 to 3 which show a medicament dispenser 1 which comprises a body 10 that defines a dosing chamber 20 in its interior, as shown in the cross-sectional view of FIG. 4.

The body 10 comprises an inlet 11 that is configured to interface with a syringe 70 and an outlet 15 that is configured to interface with a needle 60, as shown by way of example in FIG. 4.

The dosing chamber 20 has a dosing chamber inlet 21 that is fluidly connected to the inlet 11 of the body 10, for example by an inlet passage 13, and a dosing chamber outlet 22 that is fluidly connected to the outlet 15 of the body 10, for example by an outlet passage 16. The inlet passage 13 and the outlet passage 16 may be straight.

The dosing chamber 20 may be cylindrical. It may have a circular cross-section in a plane perpendicular to its longitudinal axis. Other cross-section shapes may be provided, such as square or hexagonal. The dosing chamber 20 may be straight, as shown in FIG. 4. The dosing chamber 20 may have the shape (excluding the portions immediately adjacent the dosing chamber inlet 21 and dosing chamber outlet 22) of a right-circular cylinder.

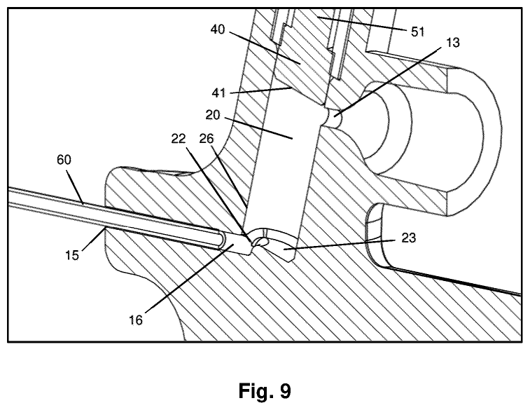

The dosing chamber inlet 21 may be provided towards one end of the dosing chamber 20 that may be proximal the location of the inlet 11. The dosing chamber outlet 22 may be provided towards an opposite end of the dosing chamber 20 that is distal the location of the inlet 11. In an embodiment, the dosing chamber outlet 22 is located very close to or at a terminal end face 23 of the dosing chamber 20 as shown in FIG. 9. For example, the dosing chamber outlet 22 may be formed in the terminal end face 23 and/or at a junction between the terminal end face 23 and a side wall 26 of the dosing chamber 20.

The terminal end face 23 may smoothly transition to the outlet passage 16, for example without any abrupt changes in curvature or re-entrant features that might hinder air bubble movement. The dosing chamber outlet 22 may be provided with a funnel portion inbetween the terminal end face 23 and the outlet passage 16.

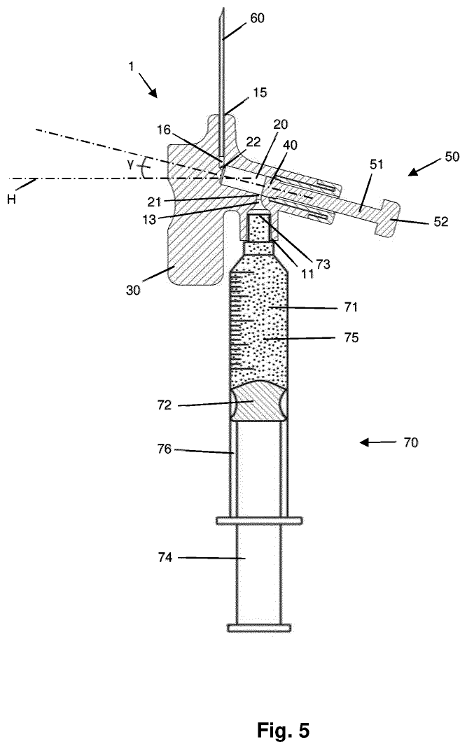

The dosing chamber outlet 22 may be located at a periphery of the terminal end face 23, for example, immediately adjacent the side wall 26. Thus, when the medicament dispenser is orientated with the outlet 15 of the body 10 pointing vertically upwards, as shown in FIG. 5, the dosing chamber outlet 22 may be located at the highest point of the dosing chamber 20.

The dosing chamber 20 may have an internal diameter of less than or equal to 4.0 mm, optionally less or equal to 3.5 mm, optionally less or equal to 3.0 mm. An internal diameter d₁ of the dosing chamber may be less than an internal diameter d₂ of a medicament-containing chamber 71 of the syringe 70.

The dosing chamber 20 may have, for example, a volume of between 0.03 ml to 0.06 ml.

A piston 40 is configured to be movable within the dosing chamber 20 to deliver the fixed volume of medicament out of the outlet 15 of the body 10. The piston 40 may be operatively connected to a plunger 50 that may extend outside the body 10 of the medicament dispenser 1.

As most clearly seen in FIGS. 1 to 3, the body 10 may comprise a front portion 31, in which the outlet 15 may be provided, and a finger grip 30 that may extend rearwardly from the front portion 31. The finger grip 30 may be relatively thin. A concave portion 32 may be provided on each side of the body 10 to provide a resting zone for a finger or thumb of a user.

The body 10 may further comprise a rear extension 34 that may define the inlet 11. The rear extension 34 may be tubular. The rear extension 34 may extend above and be spaced from the finger grip 30. The body 10 may additionally comprise an upper extension 33 that that may extends upwardly from the front portion 31 and may receive the plunger 50. The upper extension 33 may be tubular and may be angled backwardly towards the inlet 11 of the body 10 such that the plunger 50 is also angled backwardly towards the inlet 11.

The body 10 may comprise a viewing window 35 to allow visual inspection of the interior of the dosage chamber 20. A viewing window 35 may be provided on one or both sides of the body 10. The or each viewing window 35 may be located in the upper extension 33.

The inlet 11 may be shaped and sized to receive in a fluid-tight manner the syringe 70. The syringe 70 may be interfaced with the inlet 11 with or without a needle being mounted on a barrel 76 of the syringe 70. The inlet 11 of the body 10 and in particular the rear extension 34 may be configured as a Luer lock for interfacing with the syringe 70. It will be understood that other interfaces may be provided as needed to interface with a particular design of syringe.

The outlet 15 may be shaped and sized to receive in a fluid-tight manner the needle 60 as shown in FIGS. 1 to 3. The needle 60 may extend into the outlet passage 16. The needle 60 may be permanently attached to the outlet 15 before the medicament dispenser 1 is provided to the user. Alternatively, the needle 60 may be connected to the outlet 15 by the user but thereafter not be intended to be removed. In a further alternative the needle 60 may be connectable and removable from the outlet 15.

The plunger 50 may be slidably received in the upper extension 33. The plunger 50 may comprise a plunger cap 52 that is connected to the piston 40 in the dosing chamber 20 by a rod 51. The plunger 50 may be user-operable. The plunger 50 may be manually operable, for example by applying pressure to the plunger cap 52. It may be sized and configured to enable one-handed holding of the body 10 of the medicament dispenser 1 using two or more digits of one hand, for example gripping the finger grip 30, and activation of the plunger 50 using one or more digits of the same hand.

Thus, the piston 40 of the medicament dispenser 1 may be moved by a user-applied force. In some embodiments the piston 40 may be moved by a manually-applied force. In other embodiments, the piston 40 may not be actuated by a user-applied force but instead by using another source of motive power. An alternative source of motive power may be provided within or outside the medicament dispenser 1 for moving the piston 40 on demand. The source of motive power may comprise or consist of a source of stored energy.

A trigger may be applied for controlling the application of the motive power to the piston 40. The trigger may comprise, for example, a user-actuated button, switch or equivalent or a user action, for example pushing, sliding, pressing, compressing, etc. a component.

In some embodiments, the source of motive power may comprise or consist of a mechanical element or elements that may be triggered to as to move the piston 40 on demand. The mechanical element or elements may comprise an energy storage component that is actuated by a trigger. Operation of the trigger may release energy from the energy storage component that may then be applied to the piston 40 to move the piston 40. For example, the mechanical element or elements may comprise a spring element as the energy storage component, for example a compression spring, for example a helical compression spring that may be held in a compressed state to store energy. The spring element, once triggered, may apply the stored energy of the compressed spring element to the piston 40, directly or indirectly via one or more other components, during the expansion of the spring element so as to drive the piston 40 to move along the dosing chamber 20.

In some embodiments the source of motive power may comprise or consist of a propellant. The propellant may comprise, for example, a volatile propellant, for example the propellant may be one that boils at a predetermined temperature which in all cases should be below the local operating temperature of the medicament dispensing system during use. A suitable propellant is or includes a hydrofluoroalkane (HFA), for example HFA 134a, HFA 422d, or HFA 507c. The propellant may be stored in a compressed state in a propellant chamber isolated from the piston 40. The compressed propellant may act as an energy storage component. A trigger may be provided for releasing the propellant from the propellant chamber into fluid communication with the piston 40 (or into fluid communication with one or more components coupled to the piston 40). A valve may be provided for controlling discharge of the propellant from the propellant chamber. The trigger may comprise or consist of the valve or the trigger may control operation of the valve. The propellant, once released, expands and moves the piston 40 (or one or more components that themselves in turn moves the piston 40) so as to drive the piston 40 to move along the dosing chamber 20.

Beneficially, using a source of motive power that is not a user-applied force, e.g. a spring element or a propellant as discussed above, may provide a more reliable force for moving the piston 40 in the dosing chamber 20. For example, the magnitude of the force applied may be predetermined by specifying the characteristics of the source, e.g. the spring force or the identity and volume of the propellant. Additionally, the use of such sources of motive power may help to ensure that the piston 40 is stroked along the whole length of the dosing chamber 20 on actuation. For example, in the examples of a spring element or a propellant, the force is applied (directly or indirectly) to the piston 40 until the piston 40 bottoms out against the terminal end face 23 of the dosing chamber 20.

FIG. 4 shows a pre-activation position of the piston 40. In the pre-activation position an end face 41 of the piston 40 may be located immediately adjacent the dosing chamber inlet 21, e.g. above the level of, and adjacent the dosing chamber inlet 21. As will be described below, the piston 40 may be configured to move within the dosing chamber 20 from the pre-activation position, shown in FIG. 4, to an activated position to deliver the medicament 75 from the dosing chamber 20 through the outlet 15.

In a non-illustrated embodiment, the medicament dispenser 1 may be provided with a plunger lock for preventing accidental activation of the plunger 50. The plunger lock may comprise a detent on or operably-connected to the plunger 50 that catches on the body 10. The detent may be movable from a catching orientation to a non-catching orientation. Alternatively, the detent may be removable from the plunger 50. For example, the detent may form, or form part of, a tear-off element. For example, the rod 51 may be provided with a tear-off collar that interfaces with the upper extension 33 but may be torn off the rod 51 to enable relative sliding movement of the rod 51 and upper extension 33.

As shown in FIG. 4, the dosing chamber 20 may have a dosing chamber axis X. The dosing chamber axis X may be the longitudinal axis of the dosing chamber 20. The dosing chamber axis X may be angled with respect to an inlet axis Y of the dosage chamber inlet 21 and/or an axis of the inlet passage 13. The axis of the inlet passage 13 may be coincident with the inlet axis Y. The dosing chamber axis X of the dosing chamber 20 may additionally or alternatively be angled with respect to an outlet axis Z of the dosage chamber outlet 22 and/or an axis of the outlet passage 16. The axis of the outlet passage 16 may be coincident with the outlet axis Z.

An angle α between the dosing chamber axis X and the inlet axis Y may be between 45 and 85 degrees. An angle β between the dosing chamber axis X and the outlet axis Z may be between 45 and 85 degrees.

Additionally or alternatively, the dosing chamber axis X may be orientated at an angle to a needle axis of the needle 60 when the needle 60 is interfaced with the outlet 15. The needle axis may be coincident with the outlet axis Z.

Additionally or alternatively, the dosing chamber axis X may be orientated at an angle to a syringe axis of the syringe 70 when the syringe 70 is interfaced with the inlet 11. The syringe axis may be coincident with the inlet axis Y

The angling of the dosing chamber 20 may allow the inlet passage 13 and the outlet passage 16 to be laterally offset from one another. The inlet passage 13 and the outlet passage 16 may be laterally offset and parallel to one another.

When the medicament dispenser 1 is orientated with the outlet 15 of the body 10 pointing vertically upwards, as shown in FIG. 5, a level of the dosing chamber outlet 22 may be above a level of the dosing chamber inlet 21. In this orientation, the dosing chamber 20 can be seen to slope upwardly, at an angle γ to the horizontal, from a location of the dosing chamber inlet 21 to a location of the dosing chamber outlet 22. The slope angle γ may be 5° to 45° to the horizontal, optionally 20° to 30° to the horizontal, H.

The inlet passage 13, dosing chamber 20 and outlet passage 16 may form a fluid transport path of the medicament dispenser 1. The fluid transport path may comprise a tortuous path. The tortuous path may, for example, have at least two turns such that the inlet 11 and outlet 15 of the medicament dispenser 1 are laterally-offset from one another.

The medicament dispenser 1 may be manufactured from a wide variety of suitable materials, as would be appreciated by a person skilled in the art. As non-limiting examples, the body 10 may be made from a thermoplastic, e.g. COP, PC PP. The material may be transparent or translucent. The rod 51 and plunger cap 52 may also be made from a thermoplastic, e.g. PP. The piston 40 may be made from a soft thermoplastic, e.g. PP or LDPE and/or a thermoplastic elastomer, e.g. TPO, TPU, TPS.

In use, the medicament dispenser 1 may be combined with a needle 60 and a syringe 70 containing a medicament 75 to form a medicament dispensing system. The medicament dispensing system may be intended for enteral or parenteral injection. For example, for intravenous, intramuscular, subcutaneous, intradermal, intratympanic, intra-articular, intraocular or intravitreal injection.

The dosing chamber 20 may be configured to contain during use a fixed volume of medicament 75 when full. The fixed volume of medicament 75 may comprise a single dose of medicament 75.

The medicament dispenser 1 may be configured as a disposable item, i.e. to be used once and then disposed of in a suitable sharps-safe manner.

In use, the medicament dispenser 1 may be used to purge air bubbles from a medicament 75 initially contained in the syringe 70 so as to prepare the medicament 75 for injection to a patient. An example process for purging air bubbles will now be described.

First, as shown in FIG. 4, a needle 60 is connected to the outlet 15 (if not already mounted) and the syringe 70 is interfaced with the inlet 11, for example by engaging the Luer lock at an outlet 73 of the syringe 70 with the Luer lock of the rear extension 34. At this initial stage a plunger 74 of the syringe 70 has not been activated such that the syringe piston 72 is distal the outlet 73 and all of the medicament 75 is contained in the medicament-containing chamber 71 of the syringe 70.

Next, the medicament dispenser 1 is orientated as shown in FIG. 5, with the needle pointing vertically upwards so that the dosing chamber 20 slopes upwards.

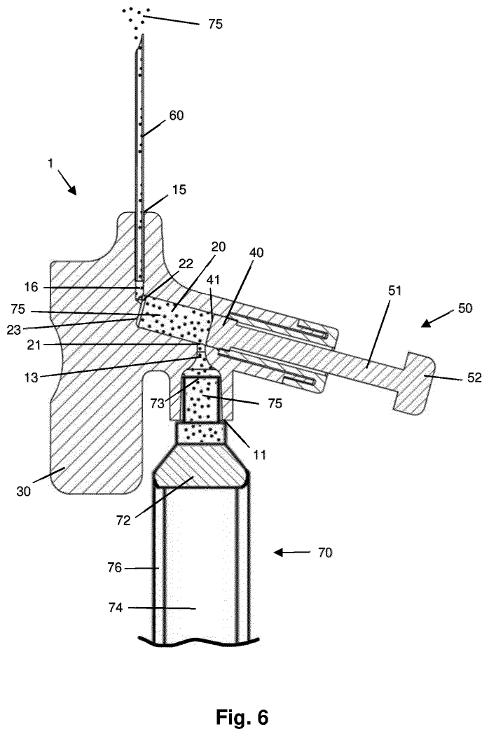

Next, as shown in FIG. 6, the plunger 74 of the syringe 70 is activated to displace medicament 75 from the medicament-containing chamber 71 out of the outlet 73 of the syringe 70 and into, through and out of the fluid transport path of the medicament dispenser 1. In other words, medicament 75 flows through the inlet passage 13, dosing chamber 20, outlet passage 16 and a portion of the medicament 75 is discharged out of the outlet 15. During this step, air bubbles in the syringe 70 are moved through the fluid transport path and out of the outlet 15. The slope angle γ of the dosing chamber 20 encourages any air bubbles entering the dosing chamber 20 to move along the dosing chamber 20 and out of the dosing chamber outlet 22.

As shown in FIG. 6, the step optionally includes fully stroking the plunger 74 of the syringe 70 so that the syringe piston 72 is displaced all the way towards the outlet 73 of the syringe 70, thus ensuring removal of substantially all air from the syringe 70.

At the end of this purging step, the dosing chamber 20 is full of medicament 75 which is substantially free of air bubbles.

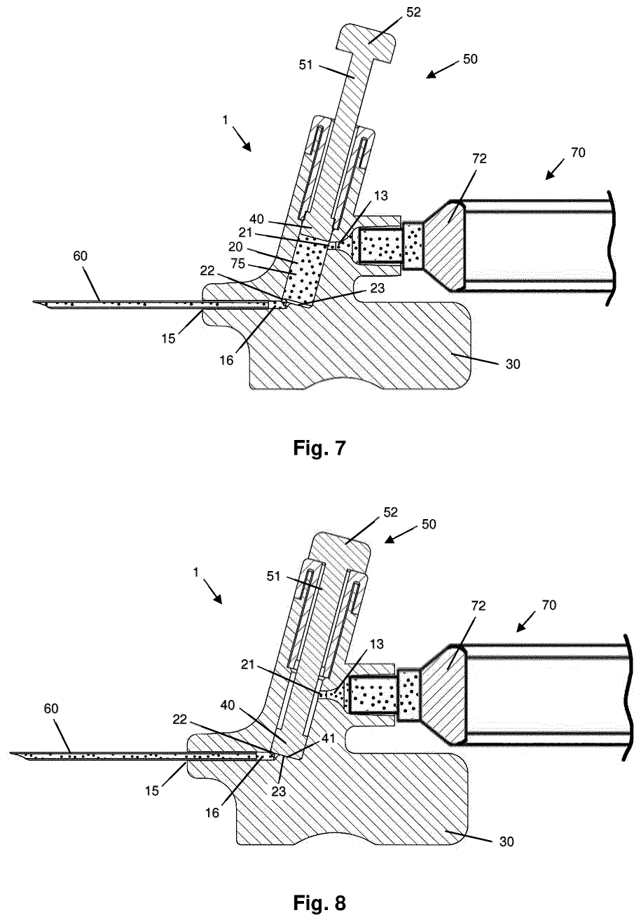

The medicament dispenser 1 may now be used to perform an injection of the medicament 75 contained in the dosing chamber 20 to a patient. As shown in FIG. 7, before injection the plunger 50 and its piston 40 are in the pre-activation position. After positioning the needle 60 at the desired injection site the plunger 50 may be activated to stroke the piston 40 along the dosing chamber 20 to discharge the required volume of medicament 75 out of the outlet 15 and needle 60. The initial movement of the piston 40 may close off the dosing chamber inlet 21 as the piston 40 sweeps over the dosing chamber inlet 21.

In an embodiment the piston 40 is fully stroked such that the position shown in FIG. 8 is obtained wherein the end face 41 of the piston 40 is located adjacent, and optionally in face-to-face contact with, the terminal end face 23 of the dosing chamber 20. Thus, the plunger 50 may be used to deliver the fixed volume of medicament 75 contained in the dosing chamber 20.

Further aspects and embodiments of the present disclosure are set out in the following clauses:
Clause A1. A medicament dispenser comprising:
   a body comprising an inlet configured to interface with a syringe and an outlet configured to interface with a needle;
   a dosing chamber within said body comprising a dosing chamber inlet fluidly connected to the inlet of the body and a dosing chamber outlet fluidly connected to the outlet of the body, the dosing chamber being configured to contain a fixed volume of medicament when full; and
   a piston configured to be movable within the dosing chamber to deliver the fixed volume of medicament out of the outlet of the body.
Clause A2. The medicament dispenser of clause A1, wherein the dosing chamber inlet is fluidly connected to the inlet of the body by an inlet passage and the dosing chamber outlet is fluidly connected to the outlet of the body by an outlet passage.
Clause A3. The medicament dispenser of clause A1 or clause A2, wherein a dosing chamber axis of the dosing chamber is angled with respect to an inlet axis of the inlet of the body and/or an outlet axis of the outlet of the body.
Clause A4. The medicament dispenser of clause A3, wherein the angle between the dosing chamber axis and the outlet axis of the body is between 45 and 85 degrees.
Clause A5. The medicament dispenser of clause A3 or clause A4, wherein the angle between the dosing chamber axis and the inlet axis of the body is between 45 and 85 degrees.
Clause A6. The medicament dispenser of any preceding clause, wherein the inlet passage and/or the outlet passage are straight.
Clause A7. The medicament dispenser of any preceding clause, wherein the inlet passage and the outlet passage are laterally offset from one another.
Clause A8. The medicament dispenser of any preceding clause, wherein, when the medicament dispenser is orientated with the outlet of the body pointing vertically upwards, a level of the dosing chamber outlet is above a level of the dosing chamber inlet.
Clause A9. The medicament dispenser of any preceding clause, wherein, when the medicament dispenser is orientated with the outlet of the body pointing vertically upwards, the dosing chamber slopes upwardly from a location of the dosing chamber inlet to a location of the dosing chamber outlet; optionally at a slope angle of 5° to 45° to the horizontal; optionally 20° to 30° to the horizontal.
Clause A10. The medicament dispenser of any preceding clause, wherein the inlet and the outlet are laterally offset from one another; and optionally are laterally offset and parallel to one another.
Clause A11. The medicament dispenser of any preceding clause, wherein the dosing chamber outlet is located in a terminal end face of the dosing chamber.
Clause A12. The medicament dispenser of any preceding clause, wherein the dosing chamber outlet is located at or adjacent a peripheral edge of a terminal end face of the dosing chamber.
Clause A13. The medicament dispenser of clause A11 or clause A12, wherein the terminal end face smoothly transitions to the outlet passage; and optionally wherein the dosing chamber outlet comprises a funnel portion inbetween the terminal end face and the outlet passage.
Clause A14. The medicament dispenser of any preceding clause, wherein the piston is configured to move within the dosing chamber from a pre-activation position to an activated position to deliver the fixed volume of medicament from the dosing chamber through the outlet.
Clause A15. The medicament dispenser of clause A14, wherein in the pre-activation position an end face of the piston is located immediately adjacent the dosage chamber inlet.
Clause A16. The medicament dispenser of clause A14 or clause A15, wherein in the activated position an end face of the piston is located in face-to-face contact with the terminal end face of the dosing chamber.
Clause A17. The medicament dispenser of any preceding clause, wherein the dosing chamber has an internal diameter of less than or equal to 4.0 mm; optionally less or equal to 3.5 mm; optionally less or equal to 3.0 mm.
Clause A18. The medicament dispenser of any preceding clause, wherein a volume of the dosing chamber is between 0.03 ml to 0.06 ml.
Clause A19. The medicament dispenser of any preceding clause, wherein the fixed volume of medicament comprises a single dose of medicament.
Clause A20. The medicament dispenser of any preceding clause, wherein the piston is operatively connected to a plunger that extends outside the body of the medicament dispenser.
Clause A21. The medicament dispenser of clause A20, wherein the plunger is manually operable; optionally wherein the body and plunger are sized and configured to enable one-handed holding of the body of the medicament dispenser using two or more digits of one hand and activation of the plunger using one or more digits of the same hand.
Clause A22. The medicament dispenser of clause A20 or clause A21, wherein the plunger is angled backwardly towards a location of the inlet of the body.
Clause A23. The medicament dispenser of any one of clauses A20 to A22, further comprising a plunger lock for preventing accidental activation of the plunger.
Clause A24. The medicament dispenser of clause A23, wherein the plunger lock comprises a detent on or operably-connected to the plunger that catches on the body.
Clause A25. The medicament dispenser of clause A24, wherein the detent is movable from a catching orientation to a non-catching orientation.
Clause A26. The medicament dispenser of clause A24, wherein the detent is removable from the plunger; optionally wherein the detent comprises a tear-off element.
Clause A27. The medicament dispenser of any preceding clause, further comprising a source of motive power for moving the piston on demand; the source of motive power comprising a source of stored energy.
Clause A28. The medicament dispenser of clause A27, further comprising a trigger for controlling the application of stored energy from the source of motive power to the piston.
Clause A29. The medicament dispenser of clause A27 or clause A28, wherein the source of motive power a spring element and/or a propellant.
Clause A30. The medicament dispenser of any preceding clause, wherein the inlet of the body comprises a Luer lock for interfacing with a syringe.
Clause A31. The medicament dispenser of any preceding clause, further comprising a needle fluidly connected to the outlet of the body.
Clause B1. A medicament dispensing system comprising the medicament dispenser of any one of clauses A1 to A31 and a syringe containing medicament, the syringe being connectable to the inlet of the body.
Clause B2. The medicament dispensing system of clause B1, further comprising a needle, the needle being connectable to the outlet of the body.
Clause B3. The medicament dispensing system of clause B1 or clause B2, wherein the syringe comprises a medicament-containing chamber having an internal diameter that is larger than an internal diameter of the dosing chamber of the medicament dispenser.
Clause C1. A method for purging air bubbles from a liquid medicament initially contained in a syringe of the type having a medicament-containing chamber having an outlet located at one end and a syringe piston for discharging the liquid medicament, the method comprising the steps of:
   a) fluidly attaching the outlet of the syringe to an inlet of a medicament dispenser; and
   b) operating the syringe piston through its full stroke to displace liquid medicament from the medicament-containing chamber out of the outlet of the syringe and into, through and out of a fluid transport path of the medicament dispenser, said fluid transport path comprising the inlet of the medicament dispenser, an internal dosage chamber and an outlet of the medicament dispenser;
   wherein the internal dosage chamber has a volume that is smaller than a volume of the medicament-containing chamber, such that on completion of the full stroke of the syringe piston a portion of the liquid medicament and a quantity of air bubbles are purged out of the outlet of the medicament dispenser, and the internal dosing chamber is left full of substantially air bubble-free liquid medicament.
Clause C2. The method of clause C1, wherein the internal dosage chamber has an internal diameter that is smaller than an internal diameter of the medicament-containing chamber.
Clause C3. The method of clause C1 or clause C2, during step b) the medicament dispenser is orientated so that the outlet points vertically upwards and the internal dosing chamber slopes upwardly from a location of a dosing chamber inlet to a location of a dosing chamber outlet.
Clause C4. The method of any one of clauses C1 to C3, wherein the fluid transport path comprises a tortuous path having at least two turns such that the inlet and outlet of the medicament dispenser are laterally-offset from one another.
Clause D1. A method of manufacturing the medicament dispenser of any one of clauses A1 to A31 or the medicament dispensing system of any one of clauses B1 to B3, wherein the body is formed by injection moulding.
Clause E1. A medicament dispenser substantially as herein described with reference to and as shown in the accompanying drawings.
Clause E2. A medicament dispensing system substantially as herein described with reference to and as shown in the accompanying drawings.

1. A medicament dispenser comprising:
a body comprising an inlet configured to interface with a syringe and an outlet configured to interface with a needle;
a dosing chamber within said body comprising a dosing chamber inlet fluidly connected to the inlet of the body and a dosing chamber outlet fluidly connected to the outlet of the body, the dosing chamber being configured to contain a fixed volume of medicament when full; and
a piston configured to be movable within the dosing chamber to deliver the fixed volume of medicament out of the outlet of the body.

2. The medicament dispenser of claim 1, wherein the dosing chamber inlet is fluidly connected to the inlet of the body by an inlet passage and the dosing chamber outlet is fluidly connected to the outlet of the body by an outlet passage.

3. The medicament dispenser of claim 1, wherein a dosing chamber axis of the dosing chamber is angled with respect to an inlet axis of the inlet of the body and/or an outlet axis of the outlet of the body.

4. The medicament dispenser of claim 3, wherein the angle between the dosing chamber axis and the outlet axis of the body is between 45 and 85 degrees; and/or wherein the angle between the dosing chamber axis and the inlet axis of the body is between 45 and 85 degrees.

5. (canceled)

6. The medicament dispenser of claim 1, wherein, when the medicament dispenser is orientated with the outlet of the body pointing vertically upwards, a level of the dosing chamber outlet is above a level of the dosing chamber inlet.

7. The medicament dispenser of claim 1, wherein, when the medicament dispenser is orientated with the outlet of the body pointing vertically upwards, the dosing chamber slopes upwardly from a location of the dosing chamber inlet to a location of the dosing chamber outlet; optionally at a slope angle of 5° to 45° to the horizontal; optionally 20° to 30° to the horizontal.

8. The medicament dispenser of claim 1, wherein the inlet and the outlet are laterally offset from one another; and optionally are laterally offset and parallel to one another.

9. The medicament dispenser of claim 1, wherein the dosing chamber outlet is located in a terminal end face of the dosing chamber.

10. The medicament dispenser of claim 1, wherein the dosing chamber outlet is located at or adjacent a peripheral edge of a terminal end face of the dosing chamber.

11. The medicament dispenser of claim 9, wherein the terminal end face smoothly transitions to the outlet passage; and optionally wherein the dosing chamber outlet comprises a funnel portion inbetween the terminal end face and the outlet passage.

12. The medicament dispenser of claim 1, wherein the piston is configured to move within the dosing chamber from a pre-activation position to an activated position to deliver the fixed volume of medicament from the dosing chamber through the outlet.

13. The medicament dispenser of claim 12, wherein in the pre-activation position an end face of the piston is located immediately adjacent the dosage chamber inlet; and/or wherein in the activated position an end face of the piston is located in face-to-face contact with the terminal end face of the dosing chamber.

14. (canceled)

15. The medicament dispenser of claim 1, wherein the dosing chamber has an internal diameter of less than or equal to 4.0 mm; optionally less or equal to 3.5 mm; optionally less or equal to 3.0 mm.

16. The medicament dispenser of claim 1, further comprising a plunger that is operatively connected to the piston; and wherein the plunger extends outside the body of the medicament dispenser.

17. The medicament dispenser of claim 16, further comprising a plunger lock for preventing accidental activation of the plunger; and optionally wherein the plunger lock comprises a detent on or operably-connected to the plunger that catches on the body.

18. (canceled)

19. A medicament dispensing system comprising the medicament dispenser of claim 1 and a syringe containing medicament, the syringe being connectable to the inlet of the body.

20. The medicament dispensing system of claim 19, wherein the syringe comprises a medicament-containing chamber having an internal diameter that is larger than an internal diameter of the dosing chamber of the medicament dispenser.

21. A method for purging air bubbles from a liquid medicament initially contained in a syringe of the type having a medicament-containing chamber having an outlet located at one end and a syringe piston for discharging the liquid medicament, the method comprising the steps of:
a) fluidly attaching the outlet of the syringe to an inlet of a medicament dispenser; and
b) operating the syringe piston through its full stroke to displace liquid medicament from the medicament-containing chamber out of the outlet of the syringe and into, through and out of a fluid transport path of the medicament dispenser, said fluid transport path comprising the inlet of the medicament dispenser, an internal dosage chamber and an outlet of the medicament dispenser;
wherein the internal dosage chamber has a volume that is smaller than a volume of the medicament-containing chamber, such that on completion of the full stroke of the syringe piston a portion of the liquid medicament and a quantity of air bubbles are purged out of the outlet of the medicament dispenser, and the internal dosing chamber is left full of substantially air bubble-free liquid medicament.

22. The method of claim 21, wherein the internal dosage chamber has an internal diameter that is smaller than an internal diameter of the medicament-containing chamber.

23. The method of claim 21, during step b) the medicament dispenser is orientated so that the outlet points vertically upwards and the internal dosing chamber slopes upwardly from a location of a dosing chamber inlet to a location of a dosing chamber outlet.

24.-25. (canceled)
